(19) United States
(12) Patent Application Publication
(10) Pub. No.: US 2012/0065359 A1
(43) Pub. Date: Mar. 15, 2012

(54) COLORANT-POLYESTERS AND METHODS OF PRODUCING COLORANT-POLYESTERS

(75) Inventors: Santiago FAUCHER, Oakavaille (CA); Kimberly D. Nosella, Mississauga (CA); Rosa M. Duque, Brampton (CA)

(73) Assignee: XEROX CORPORATION, Norwalk, CT (US)

(21) Appl. No.: 12/879,587

(22) Filed: Sep. 10, 2010

(51) Int. Cl. C08G 63/08; C12P 17/18; C12P 7/62

(52) U.S. Cl. 528/354; 435/135; 435/119

(57) ABSTRACT

An enzymatic polymerization method of covalently linking a colorant to a polyester to produce a colorant-polyester compound, the method including the steps of: (a) providing a reaction solution comprised of an ester monomer, a colorant having or functionalized to have a hydroxyl group, and an enzymatic catalyst; (b) reacting the ester monomer and the colorant using the enzymatic catalyst to produce a polymeric product, wherein the polymeric product includes a colorant-polyester compound; and (c) separating the polymeric product from the reaction solution. A colorant-polyester compound includes a colorant covalently linked to a polyester and the polyester is obtained by polymerizing a lactone.

### TECHNICAL FIELD

The present disclosure generally relates to methods to functionalize colorants to improve their stability and performance in final delivery vehicles, such as inks or toners. Specifically, the present disclosure relates to methods of covalently linking colorants to polyesters using enzymatic polymerization. Also disclosed herein are colorant-polyesters produced using the enzymatic polymerization methods.

### BACKGROUND

Inks and toners contain colorants, such as pigments and dyes. Pigments are difficult to disperse into ink or toner vehicles on account of their lack of solubility, size, tendency to aggregate and physical property differences. Thus, aggregation of the pigment in the final delivery vehicle is often a problem. Accordingly, dyes are often used instead of pigments to overcome the shortfalls of the pigments. However, dyes are expensive and add cost to the final product. Additionally, dyes can affect the material's properties based on their loading, and because of their small size can lead to depressions of glass transition temperature (Tg) or other such property modifications of inks and toners. All these negative attributes relate to the small size of the dye molecules in comparison to the polymeric or macromolecular matrix that holds them in inks and toners.

The colorants are dispersed into the polymeric matrix of inks and toners, and this dispersion is desired to remain stable, without aggregation, for some time to ensure the inks and toners function properly in a print or photocopier device. Therefore, colorants are often functionalized through chemical routes to improve their stability and performance in final delivery vehicles (inks or toners). The functionalization generally relies on grafting of short chain molecules to these colorants.

However, another approach is to graft the same or similar polymers to those of the polymeric matrix of inks or toners to the colorants. This is typically done through condensation polymerization methods. Condensation polymerization methods, however, use high temperatures, high vacuum and metal catalysts that are toxic. In particular, the preparation of polyesters by condensation polymerization methods takes several days, and relies on high temperatures (T>200° C.) and low pressures (p<1 mmHg) to drive the polymerization to completion. As condensation polymerization methods operate at temperatures of greater than 200° C. and many colorants degrade above temperatures of 150° C., condensation polymerization methods can lead to degradation of colorants and are not ideal to produce colorant-polyesters. Additionally, condensation polymerization methods are not suitable for polymerizing lactones.

### SUMMARY

The present disclosure addresses these and other needs, by providing a low temperature, metal-catalyst free enzymatic polymerization method for functionalization of colorants to produce colorant-polyester compounds, where a colorant is covalently linked to a polyester.

In embodiments, an enzymatic polymerization method of covalently linking a colorant to a polyester to produce a colorant-polyester compound, the method comprising the steps of: (a) providing a reaction solution comprised of an ester monomer, a colorant having or functionalized to have a hydroxyl group, and an enzymatic catalyst; (b) reacting the ester monomer and the colorant using the enzymatic catalyst to produce a polymeric product, wherein the polymeric product comprises a colorant-polyester compound; and (e) separating the polymeric product from the reaction solution.

In embodiments, a colorant-polyester compound comprises a colorant covalently linked to a polyester and the polyester is obtained by polymerizing a lactone.

### EMBODIMENTS

In embodiments, an enzymatic polymerization method of covalently linking a colorant to a polyester to produce a colorant-polyester compound, comprising: (a) providing a reaction solution comprising an ester monomer, a colorant having or functionalized to have a hydroxyl group, and an enzymatic catalyst; (b) reacting the ester monomer and the colorant using the enzymatic catalyst to produce a polymeric product, wherein the polymeric product comprises a colorant-polyester compound; and (c) separating the polymeric product from the reaction solution.

Reaction Solution

In embodiments, an enzymatic polymerization is accomplished by providing a reaction solution that comprises a colorant, an ester monomer and an enzymatic catalyst. The enzymatic polymerization reaction may further comprise water. The polymerization may be initiated by either water present in the reaction medium or by the hydroxyl groups present on the colorant, or both. Thus, two polyester populations may be created through this mechanism in the absence of dry solvents and monomers: one polyester population has a colorant attached to it and the other polyester population has an α-hydroxyl group, with no colorant attached to it. The polyester with an α-hydroxyl group is not colored. By adjusting the amount of water and concentration of starting materials, the ratio of colored colorant-polyester to non-colored polyester can be changed.

Colorants

Suitable colorants may be dyes, or pigments, or mixtures of dye, or mixtures of pigments and dyes, and the like.

A colorant is reactive with an ester monomer present in the reaction solution via the enzyme catalyst. This may be achieved with a colorant having a reactive end group. An example of a reactive group of a colorant is a reactive hydroxyl group.

A colorant may also be functionalized to have a reactive end group to enable the colorant to be reactive with an ester monomer. This may be achieved by functionalizing the colorant to have a reactive end group.

Examples of suitable dyes include Neozapon Red 492 (BASF); Orasol Red G (Ciba); Direct Brilliant Pink B (Oriental Giant Dyes); Direct Red 3BL (Classic Dyestuffs); Supranol Brilliant Red 3BW (Bayer AG); Lemon Yellow 6G (United Chemie); Light Fast Yellow 3G (Shaanxi); Aizen Spilon Yellow C-GNH (Hodogaya Chemical); Bernachrome Yellow GD Sub (Classic Dyestuffs); Cartasol Brilliant Yellow 4GF (Clariant); Cibanon Yellow 2GN (Ciba); Orasol Black CN (Ciba); Savinyl Black RLSN (Clariant); Pyrazol Black BG (Clariant); Morfast Black 101 (Rohm & Haas); Diaazol Black RN (ICI); Orasol Blue GN (Ciba); Savinyl Blue GLS (Clariant); Luxol Fast Blue MBSN (Pylam Products); Sevron Blue 5GMF (Classic Dyestuffs); Basacid Blue 750 (BASF), Neozapon Black X51 (BASF), Classic Solvent Black 7 (Classic Dyestuffs), Sudan Blue 670 (C.I. 61554) (BASF), Sudan Yellow 146 (C.I. 12700) (BASF), Sudan Red 462 (C.I. 26050) (BASF), C.I. Disperse Yellow 238, Neptune Red Base NB543 (BASF, C.I. Solvent Red 49), Neopen Blue FF-4012 from BASF, Lampronol Black BR from ICI (C.I. Solvent Black 35), Morton Morplas Magenta 36 (C.I. Solvent Red 172), metal phthalocyanine colorants such as those disclosed in U.S. Pat. No. 6,221,137, the disclosure of which is totally incorporated herein by reference, and the like. Polymeric dyes can also be used, such as those disclosed in, for example, U.S. Pat. No. 5,621,022 and U.S. Pat. No. 5,231,135, the disclosures of each of which are entirely incorporated herein by reference, and commercially available from, for example, Milliken & Company as Milliken Ink Yellow 869, Milliken Ink Blue 92, Milliken Ink Red 357, Milliken Ink Yellow 1800, Milliken Ink Black 8915-67, uncut Reactant Orange X-38, uncut Reactant Blue X-17, Solvent Yellow 162, Acid Red 52, Solvent Blue 44, and uncut Reactant Violet X-80.

Examples of suitable pigments include PALIOGEN Violet 5100 (commercially available from BASF); PALIOGEN Violet 5890 (commercially available from BASF); HELIOGEN Green L8730 (commercially available from BASF); LITHOL Scarlet D3700 (commercially available from BASF); SUNFAST Blue 15:4 (commercially available from Sun Chemical); Hostaperm Blue B2G-D (commercially available from Clariant); Hostaperm Blue B4G (commercially available from Clariant); Permanent Red P-F7RK; Hostaperm Violet BL (commercially available from Clariant); LITHOL Scarlet 4440 (commercially available from BASF); Bon Red C (commercially available from Dominion Color Company); ORACET Pink RF (commercially available from Ciba); PALIOGEN Red 3871 K (commercially available from BASF); SUNFAST Blue 15:3 (commercially available from Sun Chemical); PALIOGEN Red 3340 (commercially available from BASF); SUNFAST Carbazole Violet 23 (commercially available from Sun Chemical); LITHOL Fast Scarlet L4300 (commercially available from BASF); SUNBRITE Yellow 17 (commercially available from Sun Chemical); HELIOGEN Blue L6900, L7020 (commercially available from BASF); SUNBRITE Yellow 74 (commercially available from Sun Chemical); SPECTRA PAC C Orange 16 (commercially available from Sun Chemical); HELIOGEN Blue K6902, K6910 (commercially available from BASF); SUNFAST Magenta 122 (commercially available from Sun Chemical); HELIOGEN Blue D6840, D7080 (commercially available from BASF); Sudan Blue OS (commercially available from BASF); NEOPEN Blue FF4012 (commercially available from BASF); PV Fast Blue B2GO1 (commercially available from Clariant); IRGALITE Blue BCA (commercially available from Ciba); PALIOGEN Blue 6470 (commercially available from BASF); Sudan Orange G (commercially available from Aldrich), Sudan Orange 220 (commercially available from BASF); PALIOGEN Orange 3040 (BASF); PALIOGEN Yellow 152, 1560 (commercially available from BASF); LITHOL Fast Yellow 0991 K (commercially available from BASF); PALIOTOL Yellow 1840 (commercially available from BASF); NOVOPERM Yellow FGL (commercially available from Clariant); Ink Jet Yellow 4G VP2532 (commercially available from Clariant); Toner Yellow HG (commercially available from Clariant); Lumogen Yellow D0790 (commercially available from BASF); Suco-Yellow L1250 (commercially available from BASF); Suco-Yellow D1355 (commercially available from BASF); Suco Fast Yellow D1355, DI 351 (commercially available from BASF); HOSTAPERM Pink E 02 (commercially available from Clariant); Hansa Brilliant Yellow 5GX03 (commercially available from Clariant); Permanent Yellow GRL 02 (commercially available from Clariant); Permanent Rubine L6B 05 (commercially available from Clariant); FANAL Pink D4830 (commercially available from BASF); CINQUASIA Magenta (commercially available from DU PONT); PALIOGEN Black L0084 (commercially available from BASF); Pigment Black K801 (commercially available from BASF); and carbon blacks such as REGAL 330™ (commercially available from Cabot), Nipex 150 (commercially available from Degusssa) Carbon Black 5250 and Carbon Black 5750 (commercially available from Columbia Chemical), and the like, as well as mixtures thereof.

Also suitable are the colorants disclosed in U.S. Pat. No. 6,472,523, U.S. Pat. No. 6,726,755, U.S. Pat. No. 6,476,219, U.S. Pat. No. 6,576,747, U.S. Pat. No. 6,713,614, U.S. Pat. No. 6,663,703, U.S. Pat. No. 6,755,902, U.S. Pat. No. 6,590,082, U.S. Pat. No. 6,696,552, U.S. Pat. No. 6,576,748, U.S. Pat. No. 6,646,111, U.S. Pat. No. 6,673,139, U.S. Pat. No. 6,958,406, U.S. Pat. No. 6,821,327, U.S. Pat. No. 7,053,227, U.S. Pat. No. 7,381,831 and U.S. Pat. No. 7,427,323, the disclosures of each of which are incorporated herein by reference in their entirety.

In embodiments, solvent dyes are employed. An example of a solvent dye suitable for use herein may include spirit soluble dyes because of their compatibility with the ink carriers disclosed herein. Examples of suitable spirit solvent dyes include Neozapon Red 492 (BASF); Orasol Red G (Ciba); Direct Brilliant Pink B (Global Colors); Aizen Spilon Red C-BH (Hodogaya Chemical); Kayanol Red 3BL (Nippon Kayaku); Spirit Fast Yellow 3G; Aizen Spilon Yellow C-GNH (Hodogaya Chemical); Cartasol Brilliant Yellow 4GF (Clariant); Pergasol Yellow CGP (Ciba); Orasol Black RLP (Ciba); Savinyl Black RLS (Clariant); Morfast Black Conc. A (Rohm and Haas); Orasol Blue GN (Ciba); Savinyl Blue GLS (Sandoz); Luxol Fast Blue MBSN (Pylam); Sevron Blue 5GMF (Classic Dyestuffs); Basacid Blue 750 (BASF), Neozapon Black X51 [C.I. Solvent Black, C.I. 12195] (BASF), Sudan Blue 670 [C.I. 61554] (BASF), Sudan Yellow 146 [C.I. 12700] (BASF), Sudan Red 462 [C.I. 260501] (BASF), mixtures thereof and the like.

Fluorescent Colorant

The colorant may be a fluorescent colorant. Any fluorescent colorant that is capable of chemically attaching to a polyester during enzymatic polymerization may be used in the present disclosure. In accordance with the present disclosure, the fluorescent colorants produced herein are essentially colorless, i.e., prints made with the fluorescent toners blended with the fluorescent colorants on suitable selected paper substrates are not visible under normal viewing conditions. However, these fluorescent colorants may, in embodiments, become visible when exposed to light of a suitable wavelength, in embodiments ultraviolet (UV) light of a predetermined wavelength. This visibility may be imparted to the toner by the addition of the fluorescent colorants, which may be a material that only becomes visible upon exposure to UV light. In embodiments, a fluorescent colorant may be an emitting component or a component that fluoresces when exposed to UV light of a wavelength of from about 10 nanometers to about 400 nanometers, in embodiments from about 200 nanometers to about 395 nanometers of the UV spectral region.

In embodiments, suitable fluorescent colorants include, for example, 4,4′-bis(styryl)biphenyl, 2-(4-phenylstilben-4-yl)-6-butylbenzoxazole, 2-(2-hydroxyphenyl)benzothiazole, beta-methyl umbelliferone, 4,-methyl-7-dimethylaminocoumarin, 4-methyl-7-aminocoumarin, N-methyl-4-methoxy-1,8-naphthalimide, 9,10-bis(phenethynyl) anthracene, 5,12-bis(phenethynyl)naphthacene, DAYGLO INVISIBLE BLUE™ A-594-5, combinations thereof, and the like. Other suitable fluorescent agents include, for example, 9,10-diphenyl anthracene and its derivatives, N-salicylidene-4-dimethylaminoaniline, 2-(2-hydroxyphenyl)benimidazole, 2-(2-hydroxyphenyl)benzoxazole, combinations thereof, and the like.

Other various exemplary fluorescent colorants include fluorescent pigments, such as carboxylic-indenofluorenone, such as monocarboxylic-indenofluorenone and dicarboxylic-indenofluorenone, and 2-(5-hydroxylpentyl)-1H-thioxantheno[2,1,9-def]isoquinoline-1,3(2H)-dione. Fluorescent pigments also include various derivatized analogs, such as rhodamines, perylenes including C.I. Pigment Orange 43 and C.I. Pigment Red 194, perinones, squaraines, and BONA pigments such as C.I. Pigment Red 57 and C.I. Pigment Red 48.

Ester Monomers

In embodiments, the reaction solution includes an ester monomer. The ester monomer may be a cyclic ester monomer. Any appropriate cyclic ester monomer may be used in the enzymatic polymerization, such as a cyclic ester having from 5 to 16 carbon atoms, such as 6 to 15 carbon atoms, 7 to 12 carbon atoms, or 8 to 10 carbon atoms. The cyclic ester monomer may be a lactone, lactide and macrolide, cyclic carbonate, cyclic phosphate, cyclic depsipeptide or oxirane. Illustrative examples of appropriate cyclic ester monomers include lactones, such as oxacycloheptadec-10-en-2-one (available as AMBRETTOLIDE, from Penta Manufacturing Co.), omega-pentadecalactone (available as EXALTOLIDE, from Penta Manufacturing Co.), pentadecalactone, 11/12-pentadecen-15-olide (also known as pentadecenlactone), hexadecenlactone and caprolactone. Other suitable ester monomers include β-propiolactone, β-butyrolactone, propylmalolactonate, 2-methylene-4-oxa-12-dodecanolide, poly(butadiene-b-pentadecalactone, poly(butadiene-b-ε-CL), ε-caprolactone, (R) and (S)-3-methyl-4-oxa-6-hexanolide, 1,3-dioxane-2-one, 1,4-dioxane-2-one, 3(S)-isopropylmorpholine-2,5-dione, Morpholine-2,5-dione derivatives, trimethylene carbonate, 1-methyl trimethylene carbonate, 8-octanolide, 3-Decalactone, 12-Dodecanolide, α-Methylene macrolides, and α-Methylene-δ-valerolactone.

In embodiments, the reaction solution may include a non-cyclic ester monomer. Exemplary non-cyclic ester monomers include diacids, hydroxyl acids, and diesters. For example, suitable non-cyclic ester monomers that may be used include 10-hydroxy decanoic acid, 6-hydroxyhexanoic acid, 10-hydroxyhexadecanoic acid, 12-hydroxydodecanoic acid, 16-hydroxyhexadecanoic acid, 3-hydroxy butyric acid, divinyl dicarboxylates, such as divinyl adipate and divinyl sebacate, 2,2,2-trichloroethyl ester, 2,2,2-trifluoroethyl ester, unactivated diacids, such as succinic, glutaric, adipic and sebacic acids, 6-6′-O-divinyl adipate, α-ω-dixacarboxylic methyl ester, bis(hydroxylmethyl)butyric acid, and ω-fluoro-(ω-1)hydroxyl alkanoic acid.

The ester monomer may be provided to the reaction solution independently, or in the form of an organic solution comprising an ester monomer.

The molar ratio of colorant to ester monomer in the reaction solution may be any effective ratio, such as about 1:1 to about 1:50, about 1:5 to about 1:45, about 1:10 to about 1:30, about 1:15 to about 1:20, about 1:10 to about 1:20, about 1:10 to about 1:25, about 1:10 to about 1:30, about 1:10 to about 1:40, about 1:10 to about 1:50, about 1:15 to about 1:25, about 1:15 to about 1:30, about 1:15 to about 1:40, about 1:15 to about 1:50 and about 1:20 to about 1:40, about 1:20 to about 1:50. Variation in concentration of colorant to ester monomer can be used to control the molecular weight of the polymeric product.

Enzymes

The reaction solution further includes one or more appropriate enzymes. The one or more enzymes catalyze the reaction of a colorant and an ester monomer, and allow the polymerization to occur at low temperatures. An illustrative example of enzymes that can be used is a lipase, such as lipase PA, lipase PC, lipase PF, lipase A, lipase CA, lipase B (such as Candita antartica lipase B), lipase CC, lipase K, lipase MM, cutinase or porcine lipase.

The enzymes may be present in the reaction solution in immobilized or supported (non-covalently bound enzymes, such as adsorbed enzymes or enzymes that are cross-linked to other enzymes) or both immobilized and supported or free form.

The one or more enzymes may be present in the reaction solution in any effective concentration, such as from about 0.001 g/cm³ to about 0.060 g/cm³, such as from about 0.002 g/cm³ to about 0.050 g/cm³, from about 0.004 g/cm³ to about 0.040 g/cm³, from about 0.005 g/cm³ to about 0.030 g/cm³, from about 0.006 g/cm³ to about 0.020 g/cm³, from about 0.01 g/cm³ to about 0.050 g/cm³. The concentration of the one or more enzymes in the reaction solution may be controlled by varying the ratio of the mass of enzyme to the mass of an immobilizing agent, such as one or more of a cross-linked polymeric network, cross-linked polymeric beads, polymeric packings, membranes, silica-gel, silica beads, sand and zeolites.

Optional Reaction Components

The monomer may be provided to the reaction solution independently, or in the form of a monomer solution comprising monomer and a solvent. Solvents may be added to the reaction in order to help reduce the viscosity of the reaction medium to enable more facile stirring or pumping of the reaction solution.

The reaction solution may thus also comprise one or more suitable solvents, such as toluene, benzene, hexane and its analogs (such as heptane), and tetrahydrofuran and its analogs (such as 2-methyltetrahydrofuran), and methyl ethyl ketone and its analogs.

The solvent may be mixed with the monomer prior to or after addition of the monomer to the reaction solution. When present, the solvent may be of any appropriate concentration range relative to the content of monomer. For example, the solvent may comprise from 1% to about 99% of the total weight of the solvent and the cyclic monomer, such as from about 10% to about 90%, such as from about 25% to about 75%, such as from about 40% to about 60%, or such as about 50% of the total weight of the solvent and the monomer.

Reaction Conditions

The enzymatic polymerization can be undertaken at temperatures from about 50° C. to about 90° C., or from about 60° C. to about 90° C., or from about 70° C. to about 90° C., or from about 80° C. to about 90° C., or from about 50° C. to about 60° C., or from about 50° C. to about 70° C., or from about 50° C. to about 80° C., or from about 60° C. to about 80° C.

The method may be achieved through any appropriate enzymatic polymerization technique. The method may include bulk polymerization or solution polymerization, in either batch or continuous reactor configuration. In the later cases, the catalyst is packed in a column reactor and ester monomer is pumped through the catalyst to form polymer continuously. In the former case, the catalyst is added to the kettle and stirred along with the added ester monomer(s). In both cases, the colorant is added as a hydroxyl initiating site for the enzymatic polymerization. The ratio of colorant to ester monomer can be used to control the polymer molecular weight to some degree.

Bulk polymerization in a packed-bed reactor includes a reactor having one or more immobilized or supported enzymes, wherein the packed-bed reactor has an inlet and an outlet, and is fed with a solution of ester monomer and the colorant. The method may further include circulating a solution of the ester monomer and colorant through the packed-bed reactor to generate a solution enriched with colorant-polyester, such that the one or more immobilized or supported enzymes convert the ester monomers and the colorant to colorant-polymer in the packed-bed reactor during circulation, and collecting the solution enriched with colorant-polyester exiting through the outlet.

The packed-bed reactor may include one or more immobilizing agents for immobilizing the enzyme, such as a cross-linked polymeric network, cross-linked polymeric beads, polymeric packings, membranes, silica-gel, silica beads, sand and zeolites.

The reactor may be made from any appropriate material, such as stainless-steel tubing, glass tubing or polymer tubing (such as polyetheretherketone (PEEK) tubing).

The reactor may have any suitable diameter and length. In embodiments, the reactor can have an outer diameter of from about 0.1 cm to about 300 cm, such as from about 10 cm to about 100 cm, and a length of from about 1 cm to about 300 cm.

Bulk polymerization of polyesters in a continuous packed-bed reactor using immobilized enzyme catalysts is further disclosed in U.S. application Ser. No. 12/240,421, which is hereby incorporated herein in its entirety by reference.

The method may also include controlling one or more of molecular weight, polydispersity and conversion ratio of the colorant and ester monomer to colorant-polyester using one or more of residence time of the ester monomer and colorant in the reactor, dimensions of the reactor, composition of the reactor, temperature of the reactor and initiator concentration in the colorant and ester solution. Decreasing the feed rate of the reaction solution to the reactor may cause the residence time of the reaction solution within the reactor to increase, and this in turn may cause an increase in the colorant and ester monomer conversion to colorant-polyester and an increase in the molecular weight of the colorant-polyester product.

The method may include monitoring the colorant-polyester product solution collected from the outlet of the reactor to monitor the conversion of the colorant and one or more ester monomers to a colorant-polyester product. In embodiments, the monitoring includes collecting the colorant-polyester solution when the product has attained a substantially stabilized molecular weight or desired molecular weight. In embodiments, the monitoring includes collecting and analyzing the product solution to determine molecular weight of the colorant-polyester in the solution. Any suitable technique can be used for the analysis of the colorant-polyester in the solution, such as by gel permeation chromatography (GPC), differential scanning calorimetry (DSC) and nuclear magnetic resonance (NMR).

Gel permeation chromatography of the polyester utilizing a refractive index detector (RI) and a photo-diode-array (PDA) is used to confirm the covalent binding between the colorant and formed polyester. The total polyester population can be observed by the RI detector signal while the sub-population containing the colorant-polyester is observed by the PDA detector.

Following polymerization, the collected colorant-polyester may be precipitated into a solvent, such as methanol, and recovered by filtration to eliminate any residual solvent or ester monomer. The resulting cake may be extracted via any appropriate extraction, such as soxhlet extraction with methanol, to remove any unreacted colorant and unreacted ester monomer from the polyester product. Following this extraction, a polyester with covalently bound colorant and polyester is recovered from the soxhlet thimble and left to dry.

The reactor may provide in-situ filtration because the immobilized catalyst remains in the tube during the reaction, thereby avoiding the additional step of diluting and filtering of the reaction mixture after the polymerization has completed.

Reaction Products: Colorant-Polyesters and Polyesters

In embodiments, the enzymatic polyerimerization reaction produces a reaction product comprising polymeric mixture (polymeric product). The reaction product may include both colorant-polyesters and polyesters formed without covalently linked colorant (hereinafter “non-colorant polyester”). The colorant-polyester comprises a colorant and a polyester, where the colorant is covalently linked to the polyester. The colorant may be covalently linked to the polyester at an α-position.

The polyester (as a part of colorant-polyester or polyester formed without linked colorant) is formed by polymerization of ester monomers. The structure of the formed polyester is dependent on the monomer(s) used in the reaction. An exemplary structure of a polyester is expressed by the following reaction-structure model:

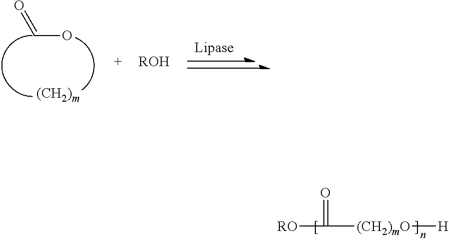

where R may be a colorant and m may be from 4 to 15. Other various structures are possible by using any of the ester monomers described above.

A non-colorant polyester may also be used for its physical, mechanical, rheological, and/or thermal properties. A non-colorant polyester may, for example, have the desired physical properties for a particular application and, at the same time, serve as a diluent (matrix) for the colorant-polyester. A non-colorant polyester therefore may help, for example, reduce the concentration of the colorant-polyester in the final product to a concentration desired for a particular application without affecting the other desired properties of the material.

In embodiments, the reaction product may include different colorant-polyesters, with different colorants linked to different polyester molecules.

The colorant-polyesters may be of any appropriate weight average molecular weight (M_{w}), such as from about 1,000 g/mol to about 50,000 g/mol, from about 2,000 g/mol to about 25,000 g/mol, from about 5,000 g/mol to about 20,000 g/mol, from about 5,000 g/mol to about 10,000 g/mol, from about 10,000 g/mol to about 15,000 g/mol, or from about 5,000 g/mol to about 25,000 g/mol.

The colorant-polyesters may be of any appropriate number average molecular weight (Mₙ), such as about 1,000 g/mol to about 50,000 g/mol, from about 2,000 g/mol to about 25,000 g/mol, from 5,000 g/mol to about 20,000 g/mol, from about 5,000 g/mol to about 10,000 g/mol, or from about 6,000 g/mol to about 8,000 g/mol.

The colorant-polyesters may be of any appropriate polydispersity index (M_{w}/Mₙ) (PDI), such as from about 1.00 to about 2.50, from about 1.25 to about 2.00, from about 1.50 to about 1.75, or from about 1.40 to about 1.60.

The colorant-polyesters may be in any structural form, such as amorphous or crystalline, depending on the types of monomers used to produce the polyester.

The colorant-polyester synthesized using a fluorescent colorant via the enzymatic polymerization retains its fluorescent behavior as observed visually and can be used as a colorant for various applications.

Applications

The polymerization method and colorant-polyesters produced by the method may have a wide range of application in various fields. In embodiments, colorant-polyesters may be used, for example, in printing, coatings, biomedical, or sensing industries. Colorant-polyesters may be used in inks or toners. For example, a fluorescent colorant-polyester could be incorporated in clear toners so that the toner could be made fluorescent for security applications. A fluorescent colorant-polyester may be incorporated in fluorescent powder coatings for automotive applications, or may be used as an additive to polymer melts for use in fluorescent clothing.

Inks and Toners

The colorant-polyesters described herein may be utilized in inks. In embodiments, the ink includes a colorant-polyester in an ink vehicle, optionally with one or more ink additives and optionally with other colorants. The colorant-polyesters described herein may be utilized in toners. In embodiments, the toner includes a colorant-polyester in a toner vehicle, optionally with one or more other colorants, optionally with one or more toner additives. The colorant-polyesters described herein may be utilized with toners produced by chemical synthesis methods, including EA toners and toners produced in suspensions, by chemical milling, combinations thereof, and the like.

In embodiments, the colorant is included in the inks or toners in an amount of from, for example, about 0.1 to about 15% by weight of the ink or toner, or from about 0.5 to about 6% by weight of the ink or toner.

Printers and Copiers

The present disclosure may be directed to a printer containing the inks described herein. Specifically, the present disclosure relates to a printer cartridge containing the inks described herein, as well as to a printer containing the printer cartridge.

The colorant-polyesters formed from the present disclosure may be used in ink jetting devices. Ink jetting devices are known in the art, and thus extensive description of such devices is not provided herein. As described in U.S. Pat. No. 6,547,380, incorporated herein by reference, ink jet printing systems generally are of two types: continuous stream and drop-on-demand.

The present disclosure may also be directed to a photocopier containing the toners described herein. Specifically, the present disclosure relates to a toner cartridge containing the toners described herein, as well as to a photocopier containing the toner cartridge.

Advantages

Enzymatic polymerization methods for producing a colorant-polyester are more environmentally friendly functionalization methods as they are undertaken at low reaction temperatures (about 50° C. to about 90° C.), without use of metal catalysts, under atmospheric pressure, and without or with a reduced amount of solvents. Additionally, the colorant-polyesters may be biodegradable. In embodiments, the enzymatic polymerization methods covalently link a colorant to a polyester at an α-position.

Enzymatic polymerization method offers a simple and effective method for functionalizing colorants. The resulting colorant-polyesters have high compatibility with the polymeric matrices that contain similar polymers, and thus, greatly simplify the incorporation of colorants into inks or toners without phase separation or precipitation, or need for dispersion. Accordingly, due to their macromolecular structure, colorant-polyesters are more likely to remain dispersed and stable in polymeric matrices of toners or inks.

As enzymatic polymerization methods do not generally require high temperatures, they do not degrade colorants by minimizing (and preventing) thermal degradation. Additionally, the low temperature used in enzymatic polymerization method is more environmentally friendly production route than condensation polymerization undertaken at 200° C. or higher. The enzymatic polymerization methods are further environmentally friendly as no metal-based catalysts and solvents are necessary and the colorant may be incorporated into biodegradable polymers produced from ester monomer.

It will be appreciated that various of the above-disclosed and other features and functions, or alternatives thereof, may be desirably combined into many other different systems or applications. Also, various presently unforeseen or unanticipated alternatives, modifications, variations or improvements therein may be subsequently made by those skilled in the art which are also intended to be encompassed by the following claims.

### EXAMPLES

### Illustrative Example 1

An illustrative example of the enzymatic polymerization reaction is shown in Reaction Diagrams I and II. A colorant may be bound to an ester monomer through enzymatic polymerization method, creating a colorant-polyester. (Reaction Diagram I)

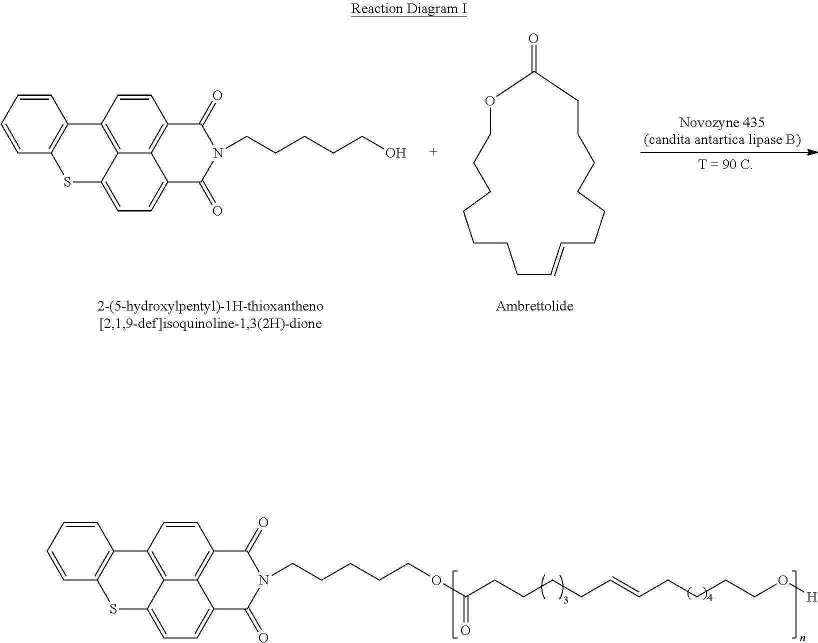

An ester monomer may also be polymerized through the enzymatic polymerization method initiated by water, optionally present in the reaction system, creating a polyester. (Reaction Diagram II)

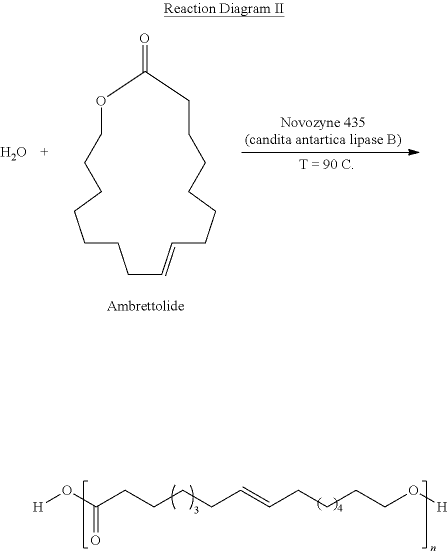

### Working Example 1

### Functionalization of Fluorescent Dye Via Enzymatic Polymerization

An example of a suitable colorant is 2-(5-hydroxylpentyl)-1H-thioxantheno[2,1,9-def]isoquinoline-1,3(2H)-dione having the below chemical formula.

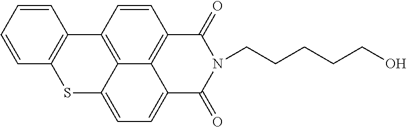

### 2-(5-hydroxylpentyl)-1H-thioxantheno[2,1,9-def]isoquinoline-1,3(2H)-dione

Reaction Diagram III below summarizes the reaction scheme for the two-part method for functionalization of colorants via enzymatic polymerization. The first part (3a) of the reaction is optional and was undertaken to attach a hydroxyl group to the colorant, if the group is not already present. In the second part (3b) of the method, the colorant with reactive hydroxyl group was covalently linked to the polyester product, wherein the hydroxyl group on the colorant, or optionally water, was the initiating site for the enzymatic polymerization.

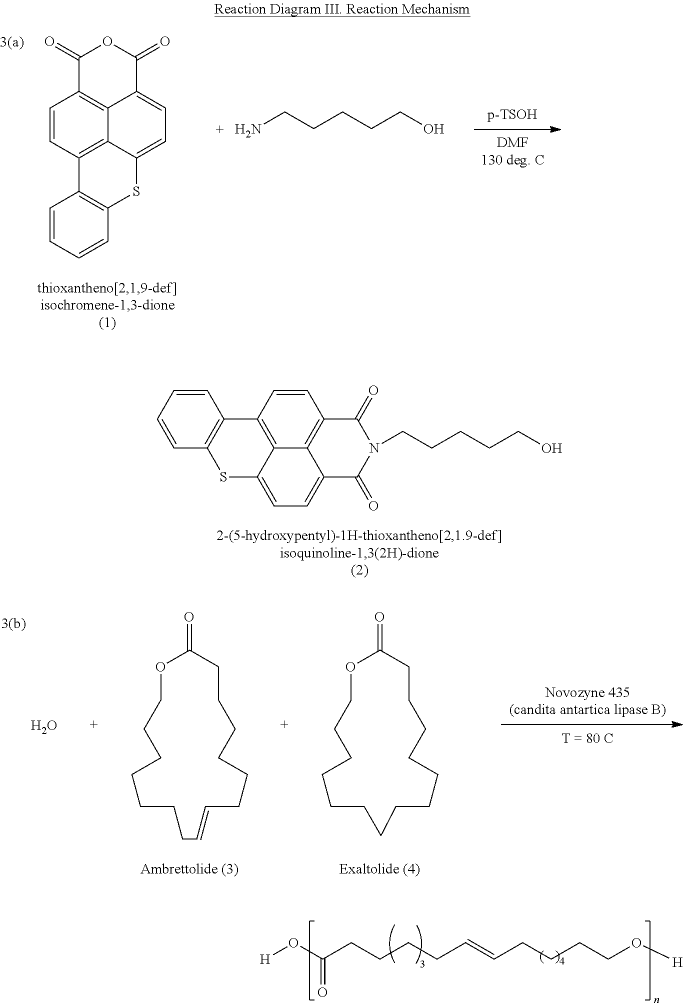

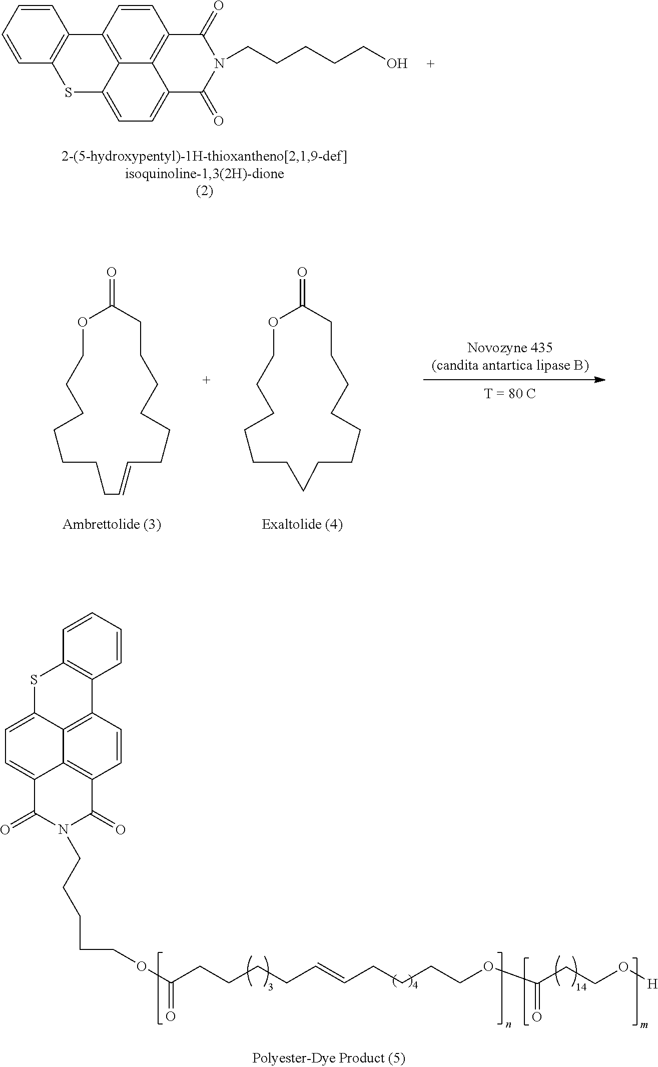

Hydroxylation of Hostasol-anhydride HYANH (3a): Thioxantheno[2,1,9-def]isochromene-1,3-dione (1) (10 g, 32.86 mmol), commercially available and marketed by Clariant as Hostasol, was used as a colorant and was loaded into a 100 ml Schlenk flask with stir bar. 5-amino-1-pentanol (20.34 g, 197 mmol) was added to the flask along with dimethylformamide (DMF) (35 ml) and p-toluene sulfonic acid (0.38 g, 2 mmol). The flask was sealed with rubber septum, purged with argon and subsequently placed in an oil bath set at 130° C. for 6 hours. The reaction was followed by thin layer chromatography (TLC) (eluent 4:1 toluene:methanol). The mixture was cooled to 50° C. and 40 ml of methanol was added to yield an orange solid, which was filtered from the solution and then washed with 200 ml of methanol. The solids were dried overnight in a vacuum oven to 12.98 g of solid product (2-(5-hydroxypentyl)-1H-thioxantheno[2,1,9-def]isoquinoline-1,3(2H)-dione) (2).

Functionalization of 2-(5-hydroxypentyl)-1H-thioxantheno[2,1,9-def]isoquinoline-1,3(2H)-dione pigment (2) with polyester chain via enzymatic polymerization (3b): Ambrettolide (3) (50 g, 198 mmol), Exaltolide (4) (47.6 g, 198 mmol), Novozyme 435 (Candita Antartica Lypase B supported on beads, 3.33 g), toluene (107.3 g) and 2-(5-hydroxypentyl)-1H-thioxantheno[2,1,9-def]isoquinoline-1,3(2H)-dione (9.64 g, 24.8 mmol) were loaded into a 250 ml glass schlenk flask along with a stir bar. The flask was sealed with a rubber septum, purged with argon, and then placed in an oil-bath preset to 80° C. so that the monomer may polymerize overnight. Following this period the flask was left to cool and the contents recovered. The solid wax-like material was then dissolved in a small amount of dichloromethane (approx. 100 ml), filtered via vacuum filtration in order to remove the catalyst, and then the filtrate was added to approximately 2 L of methanol to precipitate the polymer out of solution. The polymer precipitate was recovered via a second vacuum filtration and the retentate loaded to a soxhlet thimble. The material was then soxhlet extracted in methanol to wash the polymer precipitate over 7 days. 2-(5-hydroxypentyl)-1H-thioxantheno[2,1,9-def]isoquinoline-1,3(2H)-dione (2) is slightly soluble in methanol while the functionalized dye (5) is not due to its polymeric nature. The resulting solid material was bright orange indicating that the pigment was covalently tethered to the polyester to create a polyester-dye.

When the dye-polyester product (5) was diluted in tetrahydrofuran (THF) and injected into a size exclusion chromatograph (SEC) or gel permeation chromatograph (GPC) equipped with a photo-diode array detector (PDA), the material had a single macromolecular peak at a residence time of 41.9 minutes (with internal toluene standard with MW of 92 g/mol, reported as 94 g/mol by the GPC analysis, peak at 54.4 minutes). Based on narrow polystyrene standards, the GPC also reported that the polyester-dye product had a M_{w} of 9,770 g/mol, Mₙ of 6,460 g/mol, and PDI of 1.51. Complete polyester population (comprising polyester without linked colorant and colorant-polyester) measured by using refractive index detector (RI) had a M_{w} of 14,210 g/mol, Mₙ of 8,880 g/mol, and PDI of 1.60.

These GPC results conclusively proved that the dye was covalently bound to the polyester and that the enzymatic functionalization was successful. Furthermore, this data supports the mechanism of enzymatic polymerization that was utilized by design and outlined in Reaction Diagrams I, II and III.

The colorant-polyester synthesized using fluorescent colorant via the enzymatic polymerization retains its fluorescent behavior as observed visually and can be used as a colorant for various applications.

It will be appreciated that various of the above-disclosed and other features and functions, or alternatives thereof, may be desirably combined into many other different systems or applications. Also, various presently unforeseen or unanticipated alternatives, modifications, variations or improvements therein may be subsequently made by those skilled in the art, and are also intended to be encompassed by the following claims.

What is claimed is:

1. A colorant-polyester compound comprising a colorant and a polyester, wherein the colorant is covalently linked to the polyester and the polyester is obtained by polymerizing a lactone using an enzyme catalyst.

2. The colorant-polyester compound of claim 1, wherein the colorant is covalently linked to the polyester at an α-position.

3. The colorant-polyester compound of claim 1, wherein the colorant is a dye, a pigment, or a mixture of a pigment and a dye.

4. The colorant-polyester compound of claim 1, wherein the colorant is a fluorescent colorant selected from the group consisting of carboxylic-indenofluorenone, 2-(5-hydroxylpentyl)-1H-thioxantheno[2,1,9-def]isoquinoline-1,3(2H)-dione, rhodamine, perylene, perinone, squaraine, BONA pigment, 4,4′-bis(styryl)biphenyl, 2-(4-phenylstilben-4-yl)-6-butylbenzoxazole, 2-(2-hydroxyphenyl)benzothiazole, beta-methyl umbelliferone, 4,-methyl-7-dimethylaminocoumarin, 4-methyl-7-aminocoumarin, N-methyl-4-methoxy-1,8-naphthalimide, 9,10-bis(phenethynyl)anthracene, 5,12-bis(phenethynyl)naphthacene, DAYGLO INVISIBLE BLUE™ A-594-5,9,10-diphenyl anthracene, N-salicylidene-4-dimethylaminoaniline, 2-(2-hydroxyphenyl)benimidazole, 2-(2-hydroxyphenyl)benzoxazole, 1-[4-(2-hydroxyethoxy)-phenyl]-2-hydroxy-2-methyl-1-propane-1-one, and combinations thereof.

5. The colorant-polyester compound of claim 1, wherein the lactone is one or more members selected from the group consisting of oxacycloheptadec-10-en-2-one, pentadecalactone, pentadecenlactone, hexadecenlactone, omega-pentadecalactone, caprolactone, β-propiolactone, β-butyrolactone, propylmalolactonate, 2-methylene-4-oxa-12-dodecanolide, poly(butadiene-b-pentadecalactone, poly(butadiene-b-ε-CL), ε-caprolactone, (R) and (S)-3-methyl-4-oxa-6-hexanolide, 1,3-dioxane-2-one, 1,4-dioxane-2-one, 3(S)-isopropylmorpholine-2,5-dione, Morpholine-2,5-dione derivatives, trimethylene carbonate, 1-methyl trimethylene carbonate, 8-octanolide, δ-Decalactone, 12-Dodecanolide, α-Methylene macrolides, and α-Methylene-δ-valerolactone.

6. The colorant-polyester compound of claim 1, wherein the colorant-polyester compound has a M_{w} of about 1,000 g/mol to about 50,000 g/mol.

7. The colorant-polyester compound of claim 1, wherein the colorant-polyester compound has a Mₙ from about 1,000 g/mol to about 50,000 g/mol.

8. The colorant-polyester compound of claim 1, wherein the colorant-polyester compound has a PDI of from about 1.00 to about 2.50.

9. A method of covalently linking a colorant to a polyester to produce a colorant-polyester compound, the method comprising:
(a) providing a reaction solution comprising an ester monomer, a colorant having or functionalized to have at least one hydroxyl group, and an enzymatic catalyst;
(b) reacting the ester monomer and the colorant using the enzymatic catalyst to produce a polymeric product, wherein the polymeric product comprises a colorant-polyester compound; and
(c) separating the polymeric product from the reaction solution.

10. The method of claim 9, wherein the colorant is covalently linked to the polyester at an α-position.

11. The method of claim 9, wherein the enzymatic catalyst is one or more members selected from the group consisting of lipase PA, lipase PC, lipase PF, lipase A, lipase CA, lipase B, lipase CC, lipase K, lipase MM, cutinase and porcine lipase.

12. The method of claim 11, wherein the enzymatic catalyst is Candita antartica lipase B.

13. The method of claim 9, wherein step (b) comprises heating the reaction solution at a temperature of from about 50° C. to about 90° C.

14. The method of claim 9, wherein the ester monomer comprises from 5 to 16 carbon atoms.

15. The method of claim 9, wherein the ester monomer is a lactone.

16. The method of claim 9, wherein the colorant is a dye, a pigment, or a mixture of a pigment and a dye.

17. The method of claim 9, wherein the colorant is a fluorescent colorant selected from the group consisting of carboxylic-indenofluorenone, 2-(5-hydroxylpentyl)-1H-thioxantheno[2,1,9-def]isoquinoline-1,3(2H)-dione, rhodamine, perylene, perinone, squaraine, BONA pigment, 4,4′-bis(styryl)biphenyl, 2-(4-phenylstilben-4-yl)-6-butylbenzoxazole, 2-(2-hydroxyphenyl)benzothiazole, beta-methyl umbelliferone, 4,-methyl-7-dimethylaminocoumarin, 4-methyl-7-aminocoumarin, N-methyl-4-methoxy-1,8-naphthalimide, 9,10-bis(phenethynyl)anthracene, 5,12-bis(phenethynyl)naphthacene, DAYGLO INVISIBLE BLUE™ A-594-5,9,10-diphenyl anthracene, N-salicylidene-4-dimethylaminoaniline, 2-(2-hydroxyphenyl)benimidazole, 2-(2-hydroxyphenyl)benzoxazole, 1-[4-(2-hydroxyethoxy)-phenyl]-2-hydroxy-2-methyl-1-propane-1-one, and combinations thereof.

18. The method of claim 9, wherein the reaction solution further comprises water.

19. The method of claim 9, wherein the colorant is functionalized to have at least one hydroxyl group before step (a).

20. A colorant-polyester compound comprising a colorant covalently linked to a polyester, wherein the colorant-polyester compound is produced by the method of claim 9.
